(19) United States
(12) Patent Application Publication
(10) Pub. No.: US 2011/0229499 A1
(43) Pub. Date: Sep. 22, 2011

(54) METHOD FOR TREATMENT OF VASCULAR HYPERPERMEABILITY

(76) Inventors: Ed W. Childs, Temple, TX (US); W. Roy Smythe, Belton, TX (US)

(21) Appl. No.: 13/045,164

(22) Filed: Mar. 10, 2011

Related U.S. Application Data
(60) Provisional application No. 61/313,069, filed on Mar. 11, 2010.

(51) Int. Cl. A61K 35/00; A61K 38/19; A61K 39/00; A61K 35/16; A61P 9/00

(52) U.S. Cl. 424/184.1; 424/93.7; 514/7.6; 424/530

(57) ABSTRACT

A method for treating or preventing hemorrhagic shock comprising administering a composition comprising stem cells or a soluble factor produced by stem cells, such as stem cell factor (SCF) to a subject. For example, stem cells for use according to the invention can express elevated levels of an anti-apoptotic protein.

### REFERENCE TO RELATED APPLICATIONS

This application claims the benefit of U.S. Provisional Application No. 61/313,069 filed Mar. 11, 2010, which is incorporated herein by reference in its entirety.

This invention was made with Government support under grant nos. 5K01HL76815-3 and HL-03-011 from the National Institutes of Health. The Government has certain rights in the invention.

### INCORPORATION OF SEQUENCE LISTING

The sequence listing that is contained in the file named “SCOTP0009US_ST25.txt”, which is 5 KB (as measured in Microsoft Windows®) and was created on Mar. 4, 2011 is filed herewith by electronic submission and is incorporated by reference herein.

### BACKGROUND OF THE INVENTION

1. Field of the Invention

The invention disclosed herein is a method for treatment of vascular hyperpermeability.

2. Description of Related Art

Trauma is a leading cause of death for individuals under the age of 44. Individuals who have suffered extensive trauma exhibit hemorrhagic shock which is usually treated with fluids and medicines to maintain blood pressure. Despite the best efforts, patients die because of the inability to maintain sufficient blood pressure to properly perfuse the major organs of the body. One of the causes of death is vascular hyperpermeability secondary to hemorrhagic shock. Vascular hyperpermeability is the process by which the fluid portion of blood leaks out of the vascular structures into the tissues of the body. This leakage of fluid may cause the tissues to swell or the development of edema. Fluid congestion of the tissues and organs may develop which may thereby result in organ failure. Vascular hyperpermeability may also cause some of the fluid administered intravenously during resuscitation efforts to leak out of the vascular system and into the surrounding tissues. This leakage of the intravenous fluids from the vascular system contributes to the edema and organ failure. Leakage of intravenous fluids may also make it difficult to maintain an effective blood pressure and perfusion of the organs with oxygenated blood.

Apoptosis or programmed cell death is a normal process in which old cells die and are replaced with new cells. Apoptosis is an orderly process of cell death as distinguished from necrosis which is the result of acute cellular injury. In the body, cells are constantly dying and being replaced. Cells die when they are damaged beyond repair, infected with a virus or undergo stress, for example, starvation. These cells die, are removed, and are replaced with new cells. In some circumstances, the balance between old cell death and new cell division is out of balance. When cell division occurs at a rate faster than cell death, tumors may develop. When cell division occurs at a rate slower than cell death, a disorder or disruption in the structure and function of the affected tissue may occur.

There are several proteins involved in regulation of apoptosis. The process of apoptosis is managed by extracellular and intracellular signals. Nonlimiting examples of extracellular signals include hormones, growth factors, and cytokines, which may cross the cell membrane and thereby affect a response. The intracellular signal may be initiated by a cell under stress and may result in cell death. Before cell death can occur one or more of the signals mentioned above must be connected to the apoptotic pathway by way of regulatory proteins.

One set of proteins targets the mitochondria, as will be discussed below. The mitochondrion is a cell organelle which is essential to the life of the cell. The main function of the mitochondrion is to enable aerobic respiration, or energy production, by the cell. Disruption of the mitochondrion quickly results in cell death. The apoptotic regulatory proteins affect the permeability of the mitochondrion and cause swelling of the cell through the development of pores in the membrane. Cytochrome c is released from the mitochondrion due to the increased permeability of the outer mitochondrial membrane and serves a regulatory function as it precedes morphological changes in the cell associated with apoptosis. Once cytochrome c is released, it binds with another regulatory protein and adenosine triphosphate (ATP), which then binds to pro-caspase-9 to create an apoptosome. The apoptosome cleaves the pro-caspase to its active form of caspase-9, which in turn activates the effector, caspase-3. Caspase-3 is an enzyme which cleaves other proteins to actually start the process of intrinsic apoptosis.

Mitochondrial permeability is subject to regulation by various proteins of the Bcl-2 family of proteins. The Bcl-2 proteins are able to promote or inhibit apoptosis by either direct action on mitochondrial permeability or indirectly through other proteins. Some of the Bcl-2 proteins can stop apoptosis even if cytochrome c has been released by the mitochondrion. The Bcl-2 proteins are frequently referred to as intrinsic mitochondrial regulatory proteins.

Hemorrhagic shock and resuscitation activates a cascade of inflammatory mediators, resulting in tissue damage, multiple organ dysfunction, and if unabated, death. Ischemia associated with shock, and the resulting oxidative stress during resuscitation, contribute to the development of this systemic inflammatory response. The oxidative stress caused by ischemia/reperfusion results in an increase in reactive oxygen species (ROS) generation which activates leukocytes and damages endothelial cells. Activation of ROS that subsequently damages the endothelium has been shown to increase microvascular permeability. It has been demonstrated that ROS are generated following hemorrhagic shock. (Childs et al., 2008; Tharakan et al., 2009 and Tharakan et al., 2009). In addition, it has been shown that the endothelium is an important source of ROS generation. Since ROS are by-products of oxidative phosphorylation, most intracellular ROS are produced by the mitochondria. ROS produced at sites other than mitochondria have been reported to be involved in some apoptotic systems, but it is widely accepted that mitochondria are the predominant source of ROS produced in the “intrinsic” mitochondrial apoptotic cascade.

Apoptosis can also be regulated by certain cell-specific growth factors. For example, the endothelial cell growth factor, angiopoietin-1, has been observed to stop apoptosis and prevent vascular hyperpermeability and edema following hemorrhagic shock. The angiopoietin-1 protein prevents apoptosis of endothelial cells by regulating the apoptotic signaling pathway leading to endothelial cell death and vascular hyperpermeability (Childs et. al., 2008b). Treatment of traumatized animals with angiopoietin-1 shows that this compound is a potent inhibitor of vascular hyperpermeability and apoptosis.

If apoptosis continues to cell death, several morphological features are evident:

1. Cell shrinkage and rounding due to the breakdown of the proteinaceous cytoskeleton by enzymes.

2. The cytoplasm of the cell appears dense, and the organelles appear tightly packed.

3. Chromatin undergoes condensation into compact patches against the nuclear envelope.

4. The nuclear envelope becomes discontinuous and the DNA inside is fragmented.

5. The cell membrane shows irregular buds or blebs.

6. The cell breaks apart into several apoptotic bodies which are removed by phagocytosis.

By this process the dead and dying cells and their contents are removed in an orderly fashion and replaced with new, viable cells. There are currently no available methods or treatments to inhibit apoptosis of endothelial cells following trauma and shock. The ability to inhibit apoptosis of endothelial cells following shock would diminish conditions such as edema caused by vascular hyperpermeability resulting from the death of the endothelial cells. What is needed in the art is a method to protect the endothelial cells from apoptotic death and prevent edema from developing after the patient has suffered trauma sufficient to induce hemorrhagic shock and vascular hyperpermeability.

### SUMMARY OF THE INVENTION

In one embodiment, the invention provides a method for treating or preventing hemorrhagic shock or vascular hyperpermeability in a subject comprising administering a composition comprising an effective amount of stem cells or a soluble factor produced by stem cells to the subject. For example, in certain aspects stem cells, such as mesenchymal stem cells (MSCs) are administered to a subject. Such stem cells may be, for example, autologous stem cells, allogeneic stem cells, syngeneic stem cells or cord blood stem cells.

In certain embodiments, stem cells for use according to the invention express elevated levels of an anti-apoptotic protein, such as an anti-apoptotic Bcl family member protein (e.g., Bcl-xL, MCL-1, A-1 or Bcl-w). For instance, in some aspects, stem cells expressing elevated levels of an anti-apoptotic protein express the elevated levels from an endogenous gene. In certain aspects, however, the anti-apoptotic protein is a recombinant protein that has been introduced (e.g., transfected) into the cells or is expressed from a recombinant vector.

In further embodiments of the invention, a method is provided for treating or preventing hemorrhagic shock or vascular hyperpermeability in a subject comprising administering a composition comprising an effective amount of a soluble factor produced by stem cells to the subject. For example, the soluble factor may be a protein such as stem cell factor (SCF). A soluble stem cell protein for use according to the invention can, for example, be protein purified from a stem cells or for a stem cell media or can be produced recombinantly.

In further embodiments, compositions according to the invention comprise one or more additional components, such as a pharmaceutically acceptable excipient or carrier. In one aspect, a composition may comprise a purified or recombinant anti-apoptotic Bcl family protein, such as a Bcl-xL, MCL-1, A-1 or Bcl-w protein. For example, a composition can comprise a protein comprising the amino acid sequence of SEQ ID NO: 1, SEQ ID NO: 2 or a fragment thereof. In further aspects, a comprising may comprise an antioxidant, a mitochondrial modulator, an endothelial growth factor, or combinations thereof. Examples of antioxidants for use according the invention include, but are not limited to, ascorbic acid, glutathione, uric acid, carotenoids, α-tocopherol, ubiquinol, diprenyl, or combinations thereof. A composition may likewise comprise a mitochondrial modulator or immunomodulatory agent, such as rapamycin, Cyclosporin A, Tacrolimus or a combinations thereof.

Thus, in certain embodiments, the invention concerns compositions comprising an endothelial growth factor. An endothelial growth factor can, for example, elicit gene activation, cell proliferation, cell differentiation, matrix dissolution stimulation of regulatory cascades leading to angiogensis, cellular migration, and/or degradation of matrix metalloproteinase (MMP), or combinations thereof.

In yet a further embodiment, a method according to the invention comprises co-administering a conventional treatment for hemorrhagic shock or vascular hyperpermeability to a subject. For instance, the method can comprise administration of plasma (e.g., plasma previously harvested from the subject or from a bank) to a subject.

In still a further embodiment, the invention comprises a composition comprising stem cells, which express elevated levels of an anti-apoptotic protein. For example, the stem cells may express elevated levels of an anti-apoptotic Bcl family member protein, such as Bcl-xL, MCL-1, A-1 or Bcl-w. The anti-apoptotic protein may be expressed from an endogenous gene or may be introduced into the cells, for example, as a protein or a protein expression vector. For instance, the anti-apoptotic protein can comprise the amino acid sequence of SEQ ID NO: 1, SEQ ID NO: 2 or a fragment thereof.

In yet a further embodiment the invention provides an article of manufacture comprising stem cells, which express elevated levels of an anti-apoptotic protein. The example, the article of manufacture can be a vial, a syringe or an infusion bag.

Thus, disclosed herein is a method comprising administering in a form deliverable to a mammal a composition comprising stem cells expressing elevated levels of an anti-apoptotic protein.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: A bar graph showing the attenuation of hemorrhagic shock-induced vascular hyperpermeability by Bcl-xl administered before, during and after the onset of shock.

FIG. 2: A graph showing the attenuation of hyperpermeability induced by Bcl-xl given during resuscitation following 60 minutes of shock.

FIG. 3: A graph showing the attenuation of hyperpermeability induced by Bcl-xl given during the shock period.

FIG. 4: A graph showing the attenuation of hyperpermeability induced by Bcl-xl when given prior to the induction of shock.

FIG. 5: A bar graph showing the diminution in release of cytochrome c following administration of Bcl-xl.

FIG. 6: A bar graph showing the diminution in hemorrhagic shock-induced caspase-3 activity by Bcl-xl administration.

FIG. 7: A graph showing the elimination of vascular permeability by the administration of Cyclosporin-A prior to the onset of hemorrhagic shock.

FIG. 8: A bar graph showing the diminution in cytochrome c release following the onset of hemorrhagic shock by administration of Cyclosporin-A.

FIG. 9: A bar graph showing the diminution in hemorrhagic shock-induced caspase-3 activity by administration of Cyclosporin-A.

FIG. 10: hMSCs attenuate vascular hyperpermeability following hemorrhagic shock in an in vivo rat model.

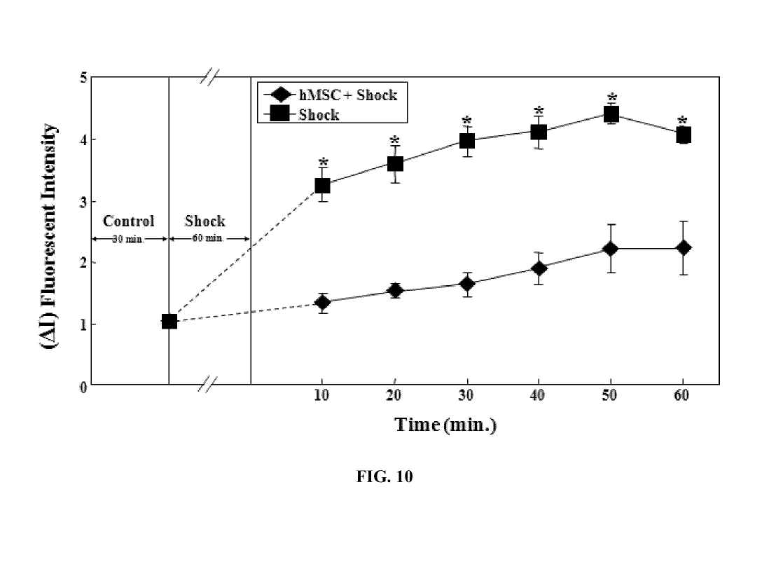

FIG. 11: A graph showing cell monolayer permeability. hMSCs attenuated BAK-induced monolayer permeability. hMSCs were grown on the lower chamber of the monolayer plate 3 days prior to growing RLMEC.

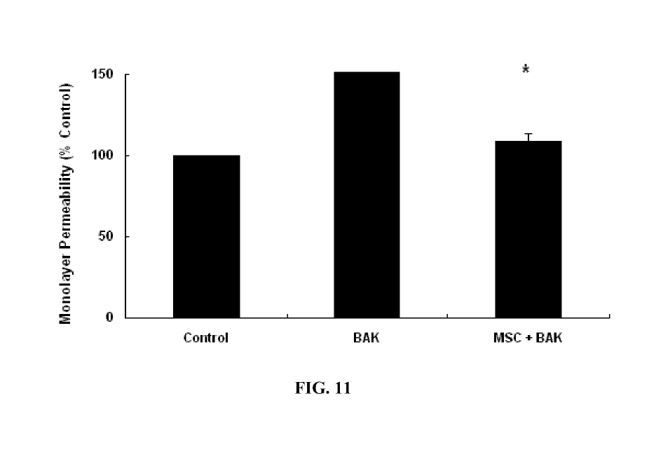

FIG. 12: A graph showing cell monolayer permeability. hMSC conditioned medium (hMSC-CM) attenuated shock serum-induced monolayer permeability (a). Regular hMSC medium (hMSC-M) has no significant effect.

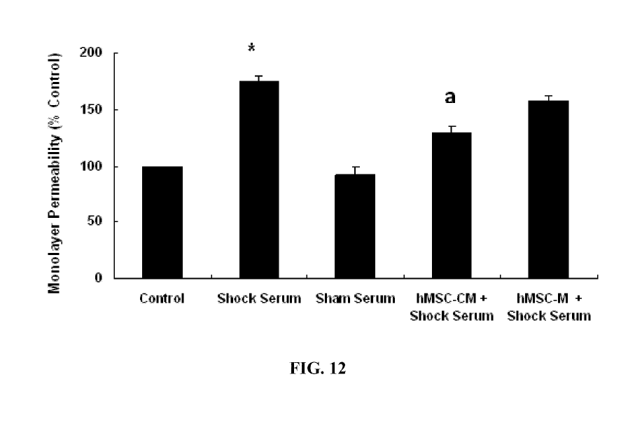

### DESCRIPTION OF ILLUSTRATIVE EMBODIMENTS

Disclosed herein is a method for treatment of vascular hyperpermeability. One of ordinary skill in the art could readily envision any number of factors, events, and/or illnesses that may result in an organism experiencing vascular hyperpermeability. Nonlimiting examples of such factors, events, and/or illnesses have been disclosed previously herein. For example, vascular permeability by any measure is dramatically increased in acute and chronic inflammation, cancer, and wound healing. This hyperpermeability is mediated by acute or chronic exposure to vascular permeabilizing agents of the type described previously herein. In an embodiment, vascular hyperpermeability is seen as a result of septic shock, closed head injury, cardiopulmonary bypass, burns, anapylaxis, direct tissue injury, ischemia-reperfusion, or combinations thereof. The disclosure hereinafter will refer to vascular hyperpermeability as a result of hemorrhagic shock however other events resulting in vascular hyperpermeability are also contemplated.

In an embodiment a method for treatment of vascular hyperpermeability comprises providing a composition comprising a stem cell having and/or expressing one or more anti-apoptotic agents and administering said composition to an organism in order to alleviate, mitigate or inhibit vascular hyperpermeability.

Hereinafter the compositions disclosed will be referred to as a stem cell composition for treatment of vascular hyperpermeability (SCV). Components of the SCV are described in more detail later herein.

In an embodiment, the SCV comprises an apoptosis-regulating protein. In an embodiment, the apoptosis-regulating protein may be provided to attenuate the intrinsic pathway leading to apoptosis to thereby reduce vascular permeability and edema associated with hemorrhagic shock, as will be discussed in greater detail herein. In another embodiment, an apoptosis-regulating protein may be provided to attenuate the extrinsic pathway leading to apoptosis to thereby reduce vascular permeability and edema associated with hemorrhagic shock, as will be discussed in greater detail herein. Hereinafter, all proteins suitable for use in this disclosure are understood to be isolated and/or purified proteins. As used herein, the terms “isolated” or “purified” protein and/or polypeptide refer to a protein and/or polypeptide which may be substantially free of other cellular material or culture medium when produced by recombinant techniques or substantially free of chemical precursors or other chemicals when chemically synthesized. As used herein, “substantially free” refers to the amount in which other components that do not adversely affect the properties of the polypeptides, compositions, and/or organisms to which the compositions are introduced may be present. For example, the proteins and/or polypeptides of the disclosed herein may be greater than about 70% pure, alternatively greater than about 75%, 80%, 85%, 90%, 95%, or 99% pure.

As used herein, the term “protein” refers to an organic compound comprising at least twenty amino acids arranged in a linear or substantially linear chain and joined by peptide bonds between the carboxyl group and the amino group of adjacent amino acid residues without regard to whether the protein was naturally or artificially synthesized and also without regard to post-translational modification of the protein, secondary, tertiary, or quaternary structure. A peptide bond is the sole covalent linkage between amino acids in the linear backbone structure of all peptides, polypeptides or proteins. The peptide bond is a covalent bond, planar in structure and chemically constitutes a substituted amide. An “amide” is any of a group of organic compounds containing the grouping —CONH—. As used herein, the term “peptide” is a compound that includes two or more amino acids linked together by a peptide bond. As used herein, the term “polypeptide” is a compound that includes three or more amino acids linked together by a peptide bond.

The apoptosis-regulating protein and/or polypeptide may be isolated and/or purified using techniques known to one of ordinary skill in the art. For example, the polypeptide may be produced from a recombinant nucleic acid. As will be understood by those of ordinary skill in the art and as used herein, a recombinant nucleic acid is a nucleic acid produced through the addition of relevant DNA into an existing organism's genome. In an embodiment, the SCV comprises an apoptosis-regulating protein which is obtained by chemical synthesis. As will be understood by those of ordinary skill in the art and as used herein, a protein may be synthesized by chemical means in a process involving the chemical ligation of peptides. Not seeking to be bound by any particular theory, a protein may be chemically synthesized via the chemical joining of amino acids. The SCV may comprise a mixture of apoptosis regulating proteins that are obtained using standard isolation and/or purification techniques and apoptosis regulating proteins obtained via chemical synthesis.

In an embodiment, the apoptosis-regulating protein comprises an intrinsic apoptosis regulatory protein. An intrinsic apoptosis regulatory protein may comprise any protein suitable for impacting the mitochondrial outer membrane permeability and thereby regulating the onset of apoptosis of endothelial cells. Not to be bound by theory, the intrinsic apoptosis regulatory protein may function to (1) reduce the mitochondrial outer membrane permeability following an event that may lead to the onset of vascular hyperpermeability, decreasing the incidence of endothelial cell apoptosis; (2) inactivate the inner mitochondrial permeability transition pore (MPTP) and prevent the formation of the mitochondrial apoptosis induced channel (MAC) which would inhibit the release of cytochrome c into the cytosol, thus preventing or lessening the occurrence of apoptosis; or both.

In an embodiment, the apoptosis-regulating protein comprises an apoptosis inhibiting protein. As used herein, “apoptosis inhibiting protein” refers to a protein which may inhibit or otherwise impede an effector molecule (e.g., another protein, a cell signaling transducer, a hormone, the like, or combinations thereof) which functions to promote the apoptotic pathway.

In an embodiment, the intrinsic apoptosis-regulating protein is an anti-apoptotic member of the Bcl-2 family of proteins. As described above, the Bcl-2 family of proteins is highly conserved, regulatory proteins for modulating the permeability of the membrane of mitochondrion. These proteins are encoded by genes located on human chromosome 13 and received their name from the cell in which they were first discovered, B cell leukemia. In an embodiment, the Bcl-2 family of proteins comprises various antiapoptotic proteins.

As used herein, “anti-apoptotic” shall mean a molecule tending to prevent or decrease the occurrence of apoptosis. Nonlimiting examples of antiapoptotic Bcl-2 proteins include, the Bcl-xL protein, the MCL-1 protein, the A-1 protein, and the Bcl-w protein. Hereinafter, anti-apoptotic Bcl-2 family members are collectively referred to as aa-Bcl2. It is contemplated that other antiapoptotic members of the Bcl-2 family not yet identified but which function to down-regulate the intrinsic apoptotic pathway may also be included in the SCV. Further, it is to be understood that other non-Bcl2 proteins that function to reduce and/or inhibit the apoptotic pathway (e.g. through attenuation of the mitochondrial outer membrane permeability) may be utilized in the SCV compositions of this disclosure. Such proteins may function to mitigate endothelial cell apoptosis and thus reduce and/or prevent the onset of vascular hyperpermeability. Such anti-apoptotic proteins may be chosen by one of ordinary skill in the art with the aid and benefit of this disclosure. The remainder of the disclosure will focus on the use aaBcl-2 proteins in the SCV although other proteins of the type described herein are also contemplated.

In an embodiment, the aa-Bcl2 comprises a polypeptide having the amino acid sequence set forth in SEQ ID NO: 1. Alternatively, the aa-Bcl2 comprises a polypeptide having the amino acid sequence identified as SEQ ID NO: 2. Hereinafter the polypeptide having the amino acid sequence set forth in SEQ ID NO:1 is referred to as human-Bcl while the polypeptide having the amino acid sequence set forth in SEQ ID NO:2 is referred to as rat-Bcl. In an embodiment the aa-Bcl2 comprises a functional derivative of human-Bcl. In an embodiment the aa-Bcl2 comprises a functional derivative of rat-Bcl.

As used herein, a “functional derivative” is a compound that possesses a biological activity (either functional or structural) that is substantially similar to the biological activity of the protein of interest (e.g., human or rat aa-Bcl). The term “functional derivatives” is intended to include the “fragments,” “variants,” “degenerate variants,” “analogs” and “homologs” or “chemical derivatives” of protein of interest (e.g., aa-Bcl). The term “fragment” is any polypeptide subset of the protein of interest (e.g., aa-Bcl). The term “variant” is meant to refer to a molecule substantially similar in structure and function to either the entire protein of interest (e.g., aa-Bcl) molecule or to a fragment thereof. A molecule is “substantially similar” to the protein of interest (e.g., aa-Bcl) if both molecules have substantially similar structures or if both molecules possess similar biological activity. Therefore, if the two molecules possess substantially similar activity, they are considered to be variants even if the structure of one of the molecules is not found in the other or even if the two amino acid sequences are not identical. The term “analog” refers to a molecule substantially similar in function to either the entire protein of interest molecule (e.g., aa-Bcl) or to a fragment thereof. The term “chemical derivative” describes a molecule that contains additional chemical moieties which are not normally a part of the base molecule. Such moieties may improve the solubility, half-life, absorption, etc of the base molecule. Alternatively the moieties may attenuate undesirable side effects of the base molecule or decrease the toxicity of the base molecule. Examples of such moieties are described in a variety of texts, such as Remington's Pharmaceutical Sciences.

It is to be understood that the present disclosure contemplates the use of any functional derivative of any protein disclosed herein. Such functional derivatives are intended to include the “fragments,” “variants,” “degenerate variants,” “analogs” and “homologs” or “chemical derivatives” of any protein described as being suitable for use in the SCV. The terms “fragments,” “variants,” “degenerate variants,” “analogs”, “homologs” and “chemical derivatives” are intended to retain their general definition as set forth previously herein with respect to the specific protein being disclosed.

In an embodiment, the SCV comprises an extrinsic apoptosis regulating protein. Such proteins may function to down-regulate the occurrence of apoptosis via mechanisms associated with the extrinsic apoptotic pathway. In some embodiments, the SCV may comprise both extrinsic apoptosis regulating proteins and intrinsic apoptosis regulating proteins.

In an embodiment, the apoptosis-regulating protein is present in the SCV in a pharmaceutically effective amount.

An apoptosis regulating protein of the type disclosed herein may be present in the formulation as an element of an integrated delivery system (IDS). The IDS may comprise a stem cell that has been genetically modified to include and/or express one or more of the apoptosis regulating proteins described previously herein. In an embodiment, the IDS comprises a stem cell.

In such an embodiment, the apoptosis regulating proteins of this disclosure may be present as an element of a vector and thus comprise a DNA vector-based apoptosis regulating protein. Vectors, including expression vectors, suitable for use in the present disclosure are commercially available and/or produced by recombinant DNA technology methods routine in the art. A vector containing an apoptosis regulating protein of the type described herein (e.g., BCl-xl) may have elements necessary for expression operably linked to such a molecule, and further can include sequences such as those encoding a selectable marker (e.g., a sequence encoding antibiotic resistance), and/or those that can be used in purification of a polypeptide (e.g., a His tag). Vectors suitable for use in this disclosure can integrate into the stem cell's cellular genome or exist extrachromosomally (e.g., an autonomous replicating plasmid with an origin of replication).

In an embodiment, the vector is an expression vector and comprises additional elements that are useful for the expression of the nucleic acid molecules of this disclosure. Elements useful for expression include nucleic acid sequences that direct and regulate expression of nucleic acid coding sequences. Elements useful for expression also can include without limitation promoters, ribosome-binding sites, introns, enhancer sequences, response elements, inducible elements that modulate expression of a nucleic acid, or combinations thereof. Elements for expression can be of bacterial, yeast, insect, mammalian, or viral origin and the vectors may contain a combination of elements from different origins. Elements necessary for expression are known to one of ordinary skill in the art and are described, for example, in Goeddel, 1990, Gene Expression Technology: Methods in Enzymology, 185, Academic Press, San Diego, Calif., the relevant portions of which are incorporated by reference herein. As used herein, operably linked means that a promoter and/or other regulatory element(s) are positioned in a vector relative to the apoptosis regulating protein in such a way as to direct or regulate expression of the molecule. An apoptosis regulating protein can be operably-linked to regulatory sequences in a sense or antisense orientation. In addition, expression can refer to the transcription of sense mRNA and may also refer to the production of protein.

In an embodiment, the apoptosis regulating proteins of the present disclosure are elements of a retroviral vector. A retroviral vector refers to an artificial DNA construct derived from a retrovirus that may be used to insert sequences into an organism's chromosomes. Adenovirus and a number of retroviruses such as lentivirus and murine stem cell virus (MSCV) are a few of the commonly used retroviral delivery systems. Adenovirus utilizes receptor-mediated infection and does not integrate into the genome for stable silencing experiments, while MSCV cannot integrate into non-dividing cell lines such as neurons, etc. A lentiviral vector is a subclass of retroviral vectors that have the ability to integrate into the genome of non-dividing as well as dividing cells. Lentiviral vectors are known in the art, and are disclosed, for example, in the following publications, which are incorporated herein by reference: Evans et al., 1999; Case et al., 1999; Uchida et al., 1998; Miyoshi et al., 1999; and Sutton et al., 1998. The lentiviral vector systems display a broad tropism and non-receptor mediated delivery. Furthermore, lentiviral vector systems have the ability to integrate into the genome for stable gene silencing, without requiring a mitotic event for integration into the genome; thus, extending its use to both dividing and nondividing cell lines. The lentiviral vector system is also not known to elicit immune responses minimizing concerns of off-target effects and use in in vivo applications.

In an embodiment the apopotosis regulating protein which is a component of an expression vector (V-ARP) has a promoter which initiates the transcription of the apoptosis regulating protein and allows for the constitutive expression of the protein. In another embodiment, the apoptosis regulating protein is operably linked to a regulatable promoter that provides inducible expression of the protein. Such inducible promoters and methods of using same are known to one of ordinary skill in the art. In an embodiment, the vector is a lentiviral vector and the markers, genes and other elements of vector may be flanked by an intact retroviral 5′ long terminal repeat (LTR) and 3′ self inactivating (SIN). Such flanking sequences are known to one of ordinary skill in the art.

The types of elements that may be included in the construct are not limited in any way and will be chosen by the skilled practitioner to achieve a particular result. For example, a signal that facilitates nuclear entry of the viral genome in the target cell, secretion of the protein by the cell, or increases the half-life of the protein may be included in the construct. It is to be understood that minor modifications of the vector as disclosed herein may be made without significantly altering the utility of the vector. As such, the description of suitable vectors is not intended to be limiting and is illustrative of one embodiment of a family of vectors.

In an embodiment the V-ARP may be delivered to cells in any way that allows the virus to infect the cell. In one embodiment, the infected cells may be used with or without further processing. In another embodiment, the infected cells may be used to infect an organism. In an embodiment, the V-ARP is introduced to a cell or cell line. Alternatively, the V-ARP is introduced to a stem cell. Herein stem cells refer to cells which are found in most, if not all, multi-cellular organisms. They are characterized by the ability to renew themselves through mitotic cell division and differentiating into a diverse range of specialized cell types. The two broad types of mammalian stem cells are: embryonic stem cells that are isolated from the inner cell mass of blastocysts, and adult stem cells that are found in adult tissues. In an embodiment, the stem cells are mesenchymal stem cells which are originally derived from the embryonal mesoderm and isolated from adult bone marrow. Mesenchymal stem cells can differentiate to form muscle, bone, cartilage, fat, marrow stroma, and tendon. During embryogenesis, the mesoderm develops into limb-bud mesoderm, tissue that generates bone, cartilage, fat, skeletal muscle and possibly endothelium. Mesoderm also differentiates to visceral mesoderm, which can give rise to cardiac muscle, smooth muscle, or blood islands consisting of endothelium and hematopoietic progenitor cells. Primitive mesodermal or mesenchymal stem cells, therefore, could provide a source for a number of cell and tissue types. A third tissue specific cell that has been named a stem cell is the mesenchymal stem cell, initially described by Fridenshtein (1982). A number of mesenchymal stem cells have been isolated (see, for example, U.S. Pat. Nos. 5,486,359; 5,827,735; 5,811,094; 5,736,396; 5,837,539; 5,837,670; 5,827,740; Jaiswal et al., 1997; Cassiede et al., 1996; Johnstone et al., 1998; Yoo et al., 1998; Gronthos, 1994; and Makino et al., 1999.

In an embodiment, a mesenchymal stem cell is modified to allow for expression of an apoptosis regulating protein of the type described herein. For example, the mesenchymal stem cell may be transfected or transduced to afford introduction of a V-ARP. Following this procedure mesenchymal stem cells containing the V-ARP may be separated from nontransfected or non-transduced cells by any appropriate methodology, such as for example by flow cytometry. The mesenchymal stem cells modified to express an apoptosis regulating protein (e.g., BCl-xl) may be further processed such that they are components of a composition that functions to inhibit, reduce, and/or prevent vascular hyperpermeability.

In an embodiment, the SCV optionally comprises one or more agents that function to attenuate the mitochondrial permeability.

In an embodiment, the SCV comprises an antioxidant. Any suitable antioxidant capable of reacting with and thereby lessening the reactivity of a ROS may be employed as the antioxidant of the SCV. In an embodiment, the SCV comprises α-lipoic acid, ascorbic acid, glutathione, uric acid, carotenoids (e.g., 3-Carotene and retinol), a-tocopherol, ubiquinol (e.g., Coenzyme Q10), deprenyl, or combinations thereof. In an embodiment, the antioxidant is present in the SCV in a pharmaceutically effective amount.

In an embodiment, the SCV comprises a mitochondrial modulator. The mitochondrial modulator may function to modulate mitochondrial membrane permeability. In some embodiments, the mitochondrial modulator is an immunomodulatory agent. Nonlimiting examples of pharmaceutical compounds suitably employed in the invention disclosed herein include Cyclosporin-A, tacrolimus (also known as FK-506, Prograf®, Adragraf® or Protopic®), other mTOR proteins, such as isrolimus (rapamycin; Rapamune®), temsirolimus (Torisel®); or combinations thereof.

Not seeking to be bound by any particular theory, the mitochondrial modulator may function to attenuate (e.g., reduce) endothelial cell apoptosis, thereby inhibiting or preventing the onset of vascular hyperpermeability. Not seeking to be bound by any particular theory, the mitochondrial modulator may decrease the response of at least a portion of the immune system of a subject to which a SCV is administered, thereby lessening the probability that the subject's immune system will reject the SCV (e.g., the protein). In an embodiment, the mitochondrial modulator is present in the SCV in a pharmaceutically effective amount.

In an embodiment, the SCV comprises a biological effector molecule. Not seeking to be bound by any particular theory, the biological effector molecule may directly or indirectly stimulate angiogenesis, that is, the growth and development of blood vessels from preexisting blood vessels, or otherwise lessen vascular hyperpermeability by contributing to vasculature proliferation. In embodiments, the biological effector molecule may comprise a molecule which will elicit biological responses including but not limited to gene activation, cell proliferation, cell differentiation, and matrix dissolution thereby leading to mitogenic activity, that is, cell division and proliferation. Such biological responses may further include stimulation of regulatory cascades leading to angiogenesis, cellular migration, and/or degradation of matrix metalloproteinase (MMP), thus leading to capillary formation.

In various embodiments, the biological effector molecule comprises a protein, a glycoprotein, a cell-surface binding molecule, a cell transport molecule, a cell-signaling molecule, a receptor molecule, a gene product, or combinations thereof. The biological effector molecule may further comprise a precursor for a protein, glycoprotein, cell-surface binding molecule, cell transport molecule, cell-signaling molecule, receptor molecule, gene product, or combinations thereof. The biological effector molecule may further comprise a transcriptional enhancer for a protein, glycoprotein, cell-surface binding molecule, cell transport molecule, cell-signaling molecule, receptor molecule, gene product, or combinations thereof.

In an embodiment, the biological effector molecule comprises an endothelial growth factor. Alternatively, the biological effector molecule comprises angiopoietin-1. Not seeking to be bound by any particular theory, angiopoietin-1 may lessen vascular hyperpermeability by disrupting the signaling pathway by which apoptosis is initiated and sustained. By disrupting the apoptotic signaling pathway, the administration of angiopoietin-1 may lessen the occurrence of apoptosis of endothelial cells and thereby lessen vascular hyperpermeability. In an embodiment, the biological effector molecule is present in the SCV in a pharmaceutically effective amount.

In an embodiment, the SCV may further comprise one or more inhibitors of the apoptotic pathway. In another embodiment, the SCV may further comprise one or more inhibitors of proapoptotic proteins such as for example BAK, BAX, and BOK.

It is contemplated that stem cells may be transfected or transduced to express one or more proteins, fragments or variants thereof that function to inhibit, reduce, and or prevent apoptosis. Consequently while the present disclosure provides description of mesenchymal stem cells expressing an aa-BCl2 protein, mesenchymal stem cells expressing other proteins that also function to inhibit apoptosis thereby mediating vascular hyperpermeability and the attendant adverse effects are contemplated for use in this disclosure. It is contemplated that in some embodiments, the SCV may comprise stem cells of the type disclosed herein that have not been modified to express elevated levels of apoptosis regulating proteins. Hereinafter the disclosure will refer to the use of stem cells genetically modified to express one or more of the apoptosis regulating proteins disclosed herein.

In an embodiment, the SCVs of this disclosure may be a component in a pharmaceutical composition wherein the composition is to be administered to an organism experiencing an undesired condition (e.g., vascular hyperpermeability) and act as a therapeutic agent for treatment of the undesired condition. Herein “treatment” refers to an intervention performed with the intention of preventing the development or altering the pathology of the undesirable condition. Accordingly “treating” refers both to therapeutic treatments and to prophylactic measures. In an embodiment, administration of therapeutic amounts of compositions of the type described herein to an organism confers a beneficial effect on the recipient in terms of amelioration of the undesirable condition. In an embodiment, the SCVs may be used in conjunction with other therapeutic methods to effect the treatment of an undesirable condition. The SCV may additionally comprise a pharmaceutically acceptable carrier or excipient. As used herein, “pharmaceutically acceptable carrier” is intended to include any and all solvents, dispersion media, coatings, antibacterial and anti-fungal agents, isotonic and absorption delaying agents, and the like, compatible with pharmaceutical administration. The use of such media and agents for pharmaceutically active substances is well known in the art.

In an embodiment, the SCV's of this disclosure may be advantageously utilized in conjunction with conventional means and methods of treating a patient experiencing or at risk for vascular hyperpermeability. For example, a conventional method for the treatment of vascular hyperpermeability may comprise the administration of fluids (e.g., plasma) to a patient experiencing hemorrhagic shock. In such an embodiment, co-administration of the SCV and blood plasma may decrease the amount of blood plasma which is necessarily administered to such a patient.

In an embodiment, a method for the treatment of vascular hyperpermeability may comprise administering a therapeutic amount of a SCV of the type described herein. Herein “therapeutic amounts” refers to the amount of the composition necessary to elicit a beneficial effect. As will be recognized by one of skill in the art, administration of the SCV may be by any suitable means. Non-limiting examples of such means of administering the composition include topical (e.g., epicutaneous), enteral (e.g., orally, via a gastric feeding tube, via a duodenal feeding tube, rectally), or parenteral, or combinations thereof. In a specific embodiment, administration of the composition may be by intravenous injection, endobronchial administration, intraaterial injection, intramuscular injection, intracardiac injection, subcutaneous injection, intraperitoneal injection, intraperitoneal infusion, transdermal diffusion, transmucosal diffusion, intracranial, intrathecal, or combinations thereof.

Although the combinations of agents comprising the SCV are described herein as a single, unitary composition, it is contemplated that these components need not be administered in the form of a single unitary compound. That is, it is hereby contemplated that components of the SCV may be administered individually or in concert. It is further contemplated that the different components of the SCV need not be administered via a single route of administration. Thus, the following disclosure is meant to apply not only in the circumstance where the components of the SCV are administered as a single, unitary composition, but also any situation in which components of the SCV are utilized in concert to for the treatment of vascular hyperpermeability. For example, in an embodiment, a first component of the vascular hyperpermeability composition may be administered to the patient shortly after the patient experiences an undesirable condition. Thereafter, the patient may be administered additional components of the SCV in subsequent time periods that may span hours, days, or weeks following the initial administration of a SCV component.

In an embodiment, the components of the SCV may be administered sequentially. In yet another embodiment, the components of the SCV may be administered simultaneously. In an embodiment, the order in which the components of the SCV are administered may be any order which will facilitate the goals or necessities of the user and depend upon a number of factors.

In an embodiment, a SCV may suitably be administered therapeutically. As used herein therapeutic administration refers to the administration of a SCV to a patient after or during a course of time in during which the patient experiences an undesirable condition. Nonlimiting examples of scenarios in which a SCV may be administered to a patient therapeutically include prior to, coincident with and/or after surgery, after a medical treatment, or following a circumstance in which the patient may have experienced some form of trauma or other disease state leading to the development of vascular hyperpermeability.

In an alternative embodiment, a SCV may suitably be administered prophylactically. As used herein, prophylactic administration refers to the administration of a SCV to a patient prior to the patient experiencing an undesired condition. Nonlimiting examples of scenarios in which a SCV may be administered to a patient prophylactically include prior to, coincident with, and/or after surgery, prior to a medical treatment, or prior to a circumstance in which the patient to whom the SCV is administered may experience some form of trauma.

In an embodiment, a SCV may suitably be administered therapeutically and prophylactically. In an embodiment, the modes of treatment described herein may be utilized at least once, alternatively multiple times, throughout the course of a treatment regime. As will be understood by those of skill in the art, the number of times a patient is administered the SCV discussed herein, as well as the dosage which is administered, may be varied to meet one or more user-desired goals or needs.

In an embodiment, an SCV of the type described herein may be administered to an organism in need thereof by any modality such as those described previously herein. In an embodiment the SVC is administered at a site proximate to the area experiencing an adverse health event. For example, the SCV may be injected at or near the site of a wound or injury.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

In one or more of the embodiments disclosed herein, the effectiveness of compositions for treating vascular hyperpermeability and methods of administering such compositions is demonstrated. The following embodiments are providing as a demonstration of the function and/or effectiveness of a one or more SCVs suitably disclosed herein.

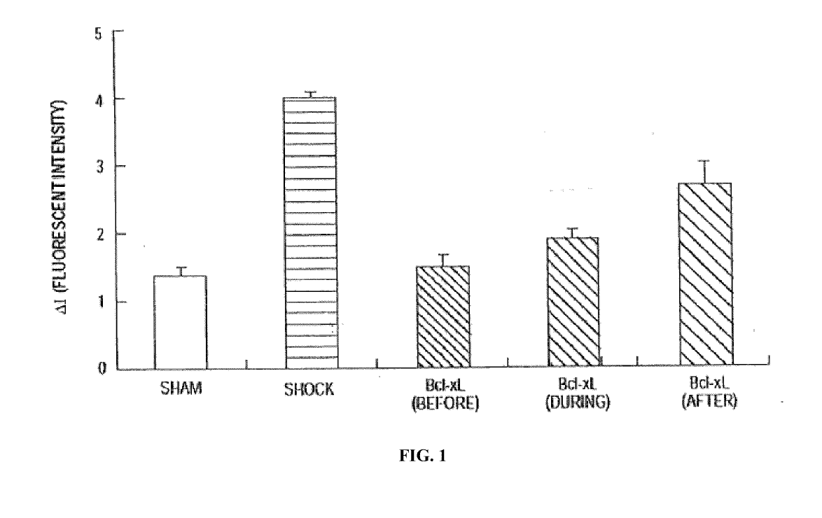

In an embodiment, a member of the Bcl family of proteins may prevent or attenuate endothelial cell dysfunction. For example, in an embodiment of the invention disclosed herein, the Bcl-2 family of proteins, and Bcl-xl in particular, is used to prevent or attenuate endothelial cell dysfunction. This attenuation of apoptosis in endothelial cells maintains the fluid barrier provided by the endothelial cells and prevents or moderates the development of edema through vascular hyperpermeability. For example, in an embodiment of the invention disclosed herein, Sprague-Dawley rats were anesthetized with urethane. Hemorrhagic shock was induced in the anesthetized rats by withdrawing blood to reduce the mean arterial pressure to 40 mm Hg for one hour. The rats were then resuscitated to 90 mmHg by administration of the shed blood and normal saline. Albumin labeled with fluorescein isothiocyanate (FITC) was given intravenously during the period in which shock was present. The mesenteric postcapillary venules in a transilluminated segment of small intestine were examined to quantitate changes in albumin flux using intravital microscopy. Recombinant Bcl-xl was suspended in a standard transfection vector and was given intravenously in an amount of approximately 2.5 microgram/ml of the total rat blood volume, before, during or after hemorrhagic shock in three separate groups of rats to determine endothelial cell integrity. Cytosolic cytochrome c levels and caspase-3 activity were also determined in mesenteric tissue collected from the animals after Bcl-xl transfection and hemorrhagic shock. As shown in FIG. 1, the administration of the protein Bcl-xl to the traumatized rats attenuated the degree of hemorrhagic shock-induced hyperpermeability. The degree of attenuation in hyperpermeability afforded by administration of Bcl-xl was greatest when Bcl-xl was administered prior to the onset of shock. Treatment of rats with Bcl-xl during the course of induced hemorrhagic shock resulted in a greater decrease in vascular hyperpermeability than did treatment with Bcl-xl after the shock period was over. A mechanism of action of the Bcl-2 family of proteins in general, and Bcl-xl, in particular, is to prevent release of cytochrome c from the mitochondrion following the onset of hemorrhagic shock. Preventing the release of cytochrome c from the mitochondria breaks the pathway to apoptosis resulting in prevention of injury to endothelial cells. Prevention of injury to endothelial cells results in an attenuation of vascular hyperpermeability during periods of hemorrhagic shock.

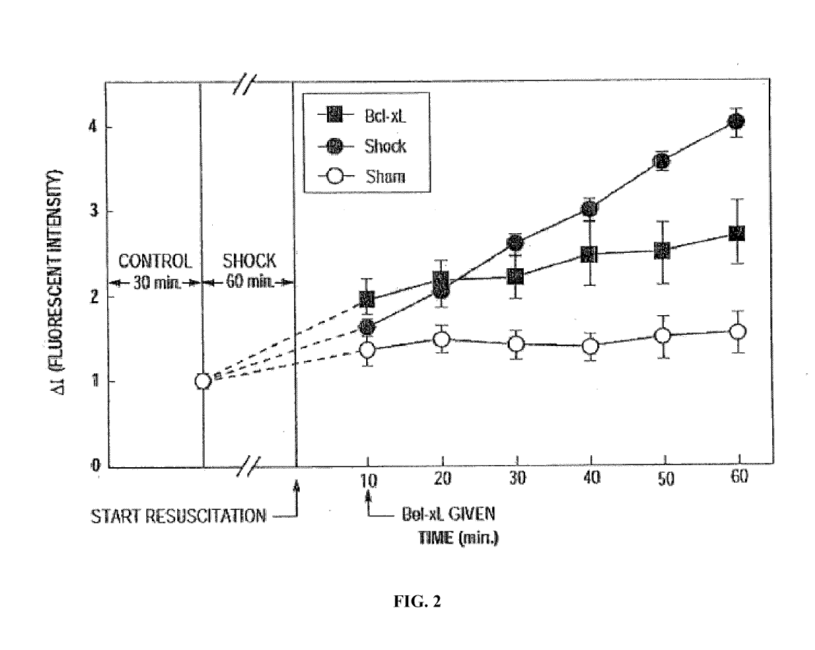

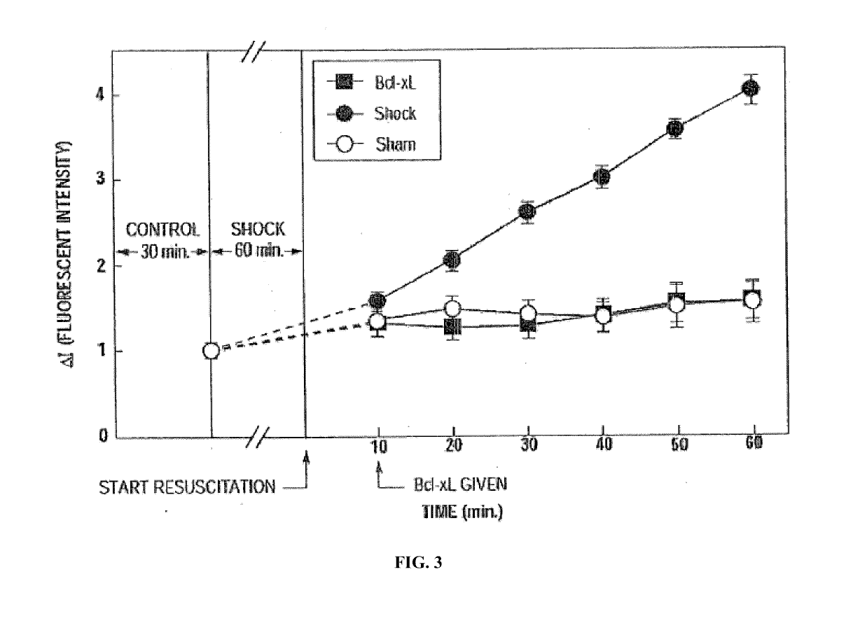

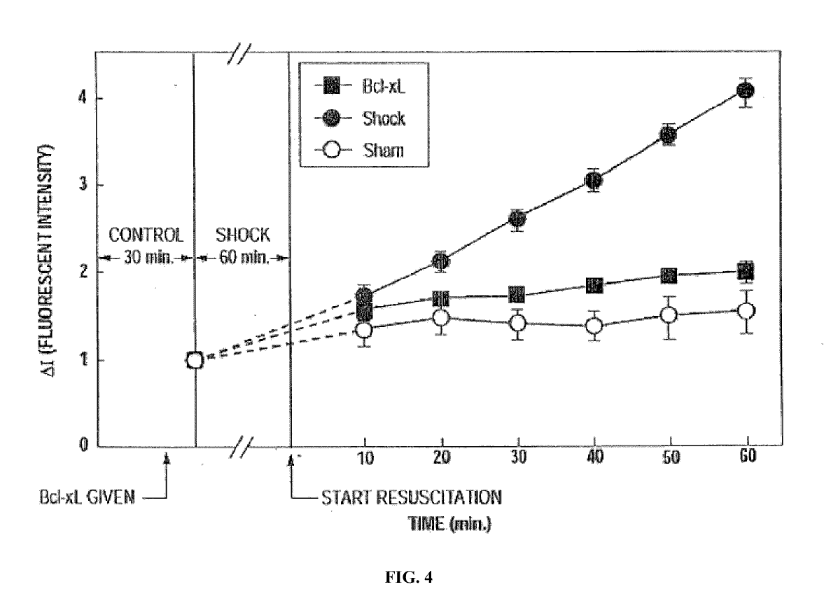

In another embodiment, a member of the Bcl family of proteins may attenuate vascular hyperpermeability. For example, in an embodiment a Bcl-xl given after one hour of shock and 10 minutes of resuscitation attenuated vascular hyperpermeability as compared to untreated animals as shown in FIG. 2. This finding confirms that intravenous administration of the intrinsic mitochondrial regulatory protein, Bcl-xl, after the onset of shock, can diminish the amount of vascular hyperpermeability. In another embodiment of the invention disclosed herein and demonstrated in FIG. 3, administration of Bcl-xl during the period of shock, but before resuscitation efforts are started, almost eliminated the hemorrhagic shock-induced hyperpermeability. In addition, Bcl-xl was given after the shock period during resuscitation and effectively reversed the hyperpermeability induced by hemorrhagic shock. These findings support the use of the intrinsic mitochondrial regulatory protein, Bcl-xl, as a “front-line” treatment of hemorrhagic shock. In yet another embodiment, hemorrhagic shock-induced hyperpermeability was almost eliminated when rats were treated with Bcl-xl prior to the onset of shock as shown in FIG. 4.

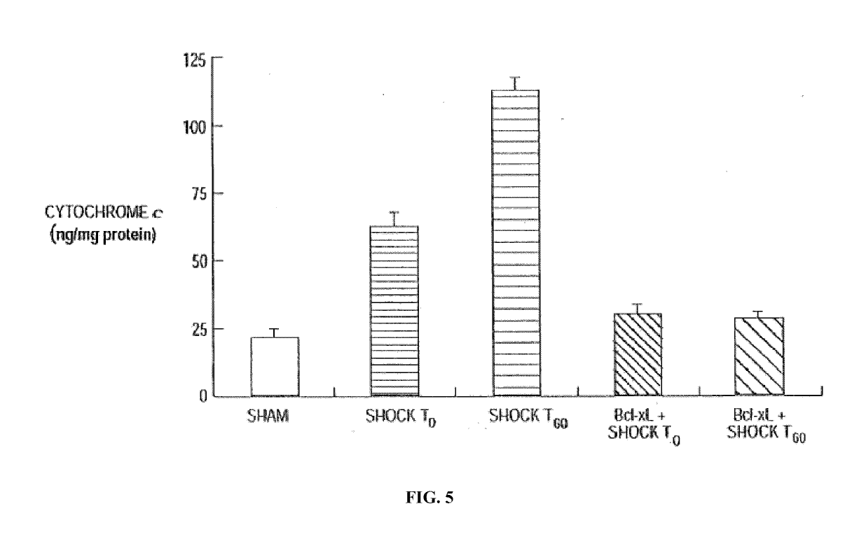

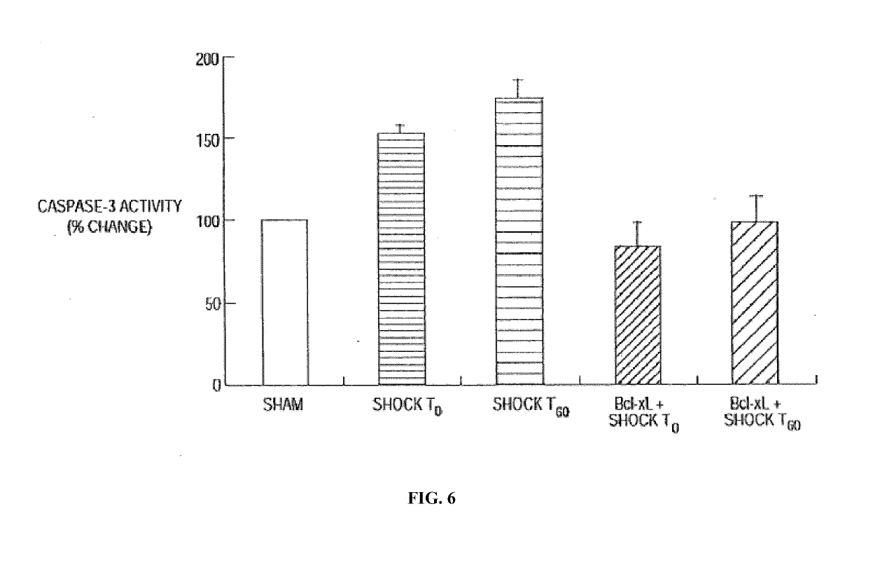

In another embodiment, a member of the Bcl family of proteins may inhibit the release of cytochrome c. For example, in another embodiment the administration of Bcl-xl inhibited the release of cytochrome c into the cytoplasm from the mitochondria following hemorrhagic shock as shown in FIG. 5. FIG. 6 demonstrates another embodiment of the invention disclosed herein. Administration of Bcl-xl reduced the activation of caspase-3 following hemorrhagic shock. As described above both cytochrome c and caspase-3 play vital roles in the regulation and initiation of apoptosis of endothelial cells following hemorrhagic shock.

In one or more of the aforementioned embodiments, Bcl-xl was disclosed as having the property of inhibiting apoptosis as measured by attenuation of vascular hyperpermeability, a decrease in cytochrome c release and reduction in caspase-3 activity following administration of Bcl-xl. The use of Bcl-xl to prevent or diminish the degree of edema following trauma in mammals is clearly indicated. The other members of the Bcl-2 family of proteins, such as BAX, BAK, MCL-1, A1 and BCL-W may also have useful properties of preventing edema as does Bcl-xl and are specifically disclosed as such, herein.

In an embodiment, the protein Bcl-xl may be administered to the test animals in the aforementioned embodiments by transfection. Standard transfection vectors, such as “transIT” and “chariot,” may be useful in facilitating entry of the intrinsic mitochondrial regulatory proteins and other substances which are disclosed herein through the membrane of the endothelial cell into the cytoplasm of the endothelial cell where regulation of apoptosis at the level of the mitochondrion can take place. The use of transfection to deliver Bcl-xl to the test animals was not meant to exclude other methods of delivery that are well known to those of ordinary skill in the art. For example, the intrinsic mitochondrial regulatory proteins could be bound to antibody or antigen-recognizing fragments of antibody which are specifically directed to receptor proteins on the cell membrane of endothelial cells. In this manner, the intrinsic mitochondrial regulatory protein could be delivered directly to the endothelial cell. Nonlimiting examples of other delivery methods include plasmid vectors, viral vectors, liposomes, antibody vectors, and others which are included in this disclosure as if specifically set forth. In an alternative embodiment, a Bcl-family protein may be administered absent a delivery vehicle.

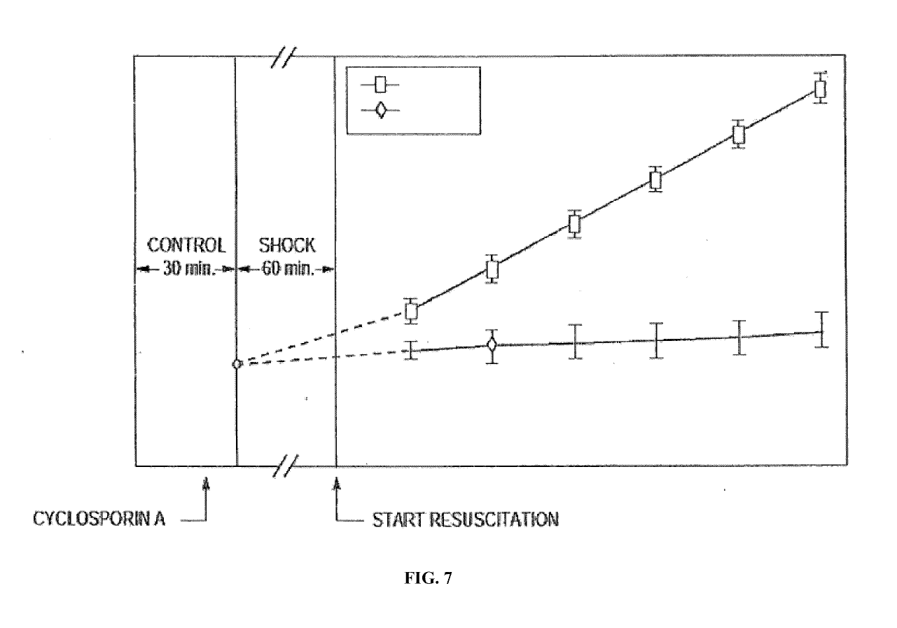

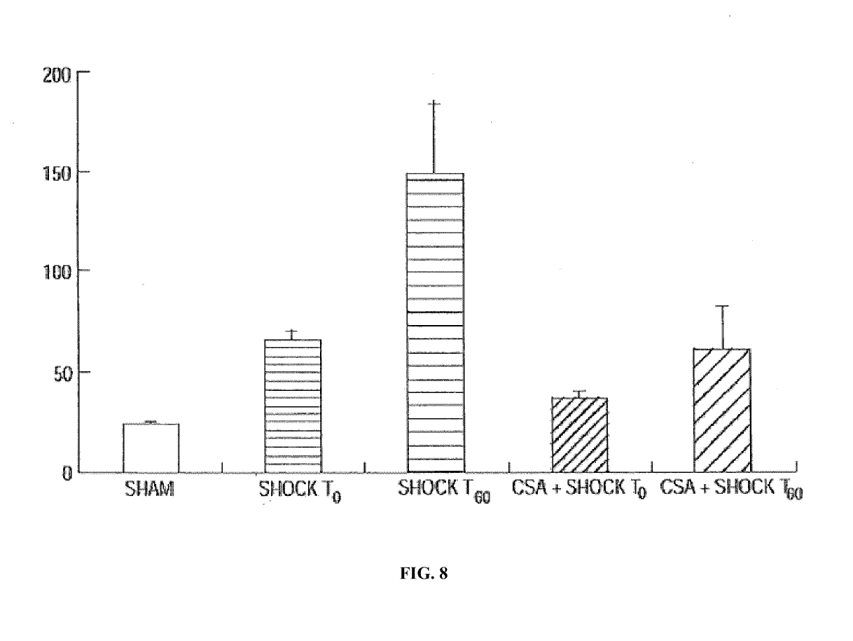

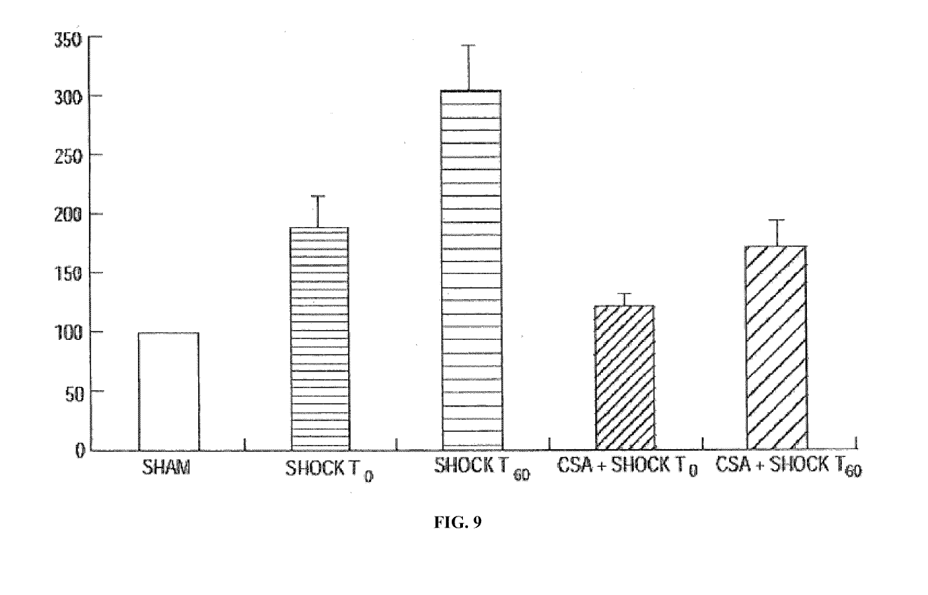

In an embodiment, other apoptotic modulators may include mediators of the immune response such as Cyclosporin-A used initially to prevent rejection of transplanted organs, also affect apoptosis of endothelial cells as shown in FIGS. 7, 8 and 9. For example, in this embodiment of the invention disclosed herein, the administration of Cyclosporin-A by transfection, for example, prior to the induction of shock in rats as described above, resulted in a complete elimination of vascular hyperpermeability as shown in FIG. 7. That Cyclosporin-A exerts its effect on vascular hyperpermeability by inhibiting apoptosis of endothelial cells is shown in FIG. 8 and FIG. 9 wherein administration of Cyclosporin-A inhibits cytochrome c release from mitochondria and diminishes the induction of caspase-3 activity by hemorrhagic shock, respectively. Cyclosporin-A is effective in preventing edema in mammals following acute trauma. The amount of Cyclosporin-A administered to traumatized animals is an amount which effectively inhibits apoptosis and is in a range of approximately 5 microliters to approximately 20 microliters per milliliter of blood volume.

Because of the role of ROS in the development of cell permeability following hemorrhagic shock, antioxidants were employed to inhibit the development of ROS and minimize the development of cell permeability and cell injury related to the development of ROS during apoptosis. In this embodiment of the invention disclosed herein, antioxidants such as alpha-lipoic acid were administered to animals traumatized as described above. The administration of alphalipoic acid attenuated the amount of vascular hyperpermeability induced by hemorrhagic shock-induced apoptosis. Alpha-lipoic acid administered by transfection in a dosage of about 100 mg/kg was effective in reducing the amount of vascular hyperpermeability if administered either before the onset of hemorrhagic shock or within 60 minutes after the development of hemorrhagic shock.

In another embodiment of the invention described herein, it is disclosed that angiopoietin-1, an endothelial cell growth factor, administered to mammals with hemorrhagic shock, attenuated the amount of vascular hyperpermeability demonstrated by those traumatized animals. Angiopoietin-1 administered intravenously in a dosage of 200 ng/ml to traumatized animals attenuated the amount of vascular hyperpermeability observed in those animals. The effect of angiopoietin-1 on lessening vascular hyperpermeability was to disrupt the apoptotic signaling mechanism which initiates and sustains the process of apoptosis by inhibiting one or a combination of factors comprising: (1) BAK peptide-induced collapse of mitochondrial transmembrane potential, (2) second mitochondrial derived activator of caspases release (smac), (3) cytochrome c release, and (4) activation of caspase-3.

As described above, intrinsic mitochondrial regulatory proteins were administered intravenously to traumatized animals. It is further disclosed herein that the intrinsic mitochondrial regulatory proteins may be administered by other routes, including, but not limited to, the sublingual route, direct injection into a body cavity or through the peritoneum into the abdominal cavity. Administration of the intrinsic mitochondrial regulatory proteins by these other avenues would raise the threshold of apoptosis and prevent vascular hyperpermeability and edema.

When foreign proteins are injected into a mammal, the host animal recognizes the proteins as foreign and attempts to eliminate them quickly from the body of the host. This rapid elimination of these administered proteins can diminish the activity of those administered proteins and deprive the host animal with their full benefit. This removal of administered proteins can be inhibited to some extent by binding to the foreign proteins substances which slow or prevent the process of natural elimination of foreign proteins. It is specifically disclosed herein, that the intrinsic mitochondrial regulatory proteins can be specifically attached to other compounds prior to administration to the traumatized animal which prolongs the effective time period in which the intrinsic mitochondrial regulatory protein can act to inhibit apoptosis in endothelial cells of traumatized animals. Those substances which can be attached to the intrinsic mitochondrial regulatory proteins to prolong their presence in the animal's circulation include but are not limited to sugars, carbohydrates, nucleotides, polyethylene glycol and the like.

The invention disclosed herein is a method for treatment of patients with edema following the development of shock. The method comprises modulating the apoptotic process in the endothelial cells lining the lumen of small venules, capillaries and other vascular structures, in order to preserve the barrier to leakage of fluid from the blood to the other tissues and prevent or diminish edema. This amelioration of edema would prevent organ failure and promote the effectiveness of resuscitation measures used to treat shock. As shown above, regulatory proteins, pharmaceuticals, antioxidants, endothelial growth factors, and other compounds and processes related to regulation of apoptosis can be modulated to prevent the death of endothelial cells and development of edema. In particular and in various embodiments, mesenchymal stem cells transfected or transduced to express elevated levels of antiapoptotic members of the Bcl-2 family of proteins, immunomodulating compounds such as Cyclosporin-A, endothelial growth factors such as angiopoietin-1, and antioxidants such as deprenyl or alpha-lipoic acid, provide such desirable results. Administration of such compounds to trauma patients, either alone or in combination, would save many lives and prevent other co-morbidities caused by the organ damage associated with edema resulting from vascular hyperpermeability. Administration of a combination of the apoptotic modulators described above would inhibit the apoptotic cascade at different points making the use of a combination of the aforementioned apoptotic modulators an effective inhibitor of vascular permeability caused by endothelial cell death. In an embodiment, a combination of apoptotic modulators suitable for use in this disclosure comprises an intrinsic regulatory protein, an immune modulator and an antioxidant. In an alternative embodiment, a combination of mesenchymal stem cells expressing apoptotic modulators suitable for use in this disclosure including without limitation an antiapoptotic protein, such as Bcl-2, Bcl-xl, MC1-1, A1 and Bcl-w, or an anti-proapoptotic protein, such as an inhibitor or antibody to a proapoptotic protein, such as BAK and BAX-1 which are combined with an immune or mitochondrial modulator, such as Cyclosporin-A, estradiol, or angiopoietin 1, and/or an antioxidant, such as deprenyl or alpha-lipoic acid.

### Example 2

The ability of mesenchymal stem cells transduced with an antiapoptotic protein to inhibit hemorraghic shock was investigated. The experimental details and results are presented in Example 3 which is attached hereto and incorporated herein.

While embodiments of the disclosure have been shown and described, modifications thereof can be made by one skilled in the art without departing from the spirit and teachings of the disclosure. The embodiments described herein are exemplary only, and are not intended to be limiting. Many variations and modifications of the disclosure disclosed herein are possible and are within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rl, and an upper limit, Ru, is disclosed, any number falling within the range is specifically disclosed. In particular, the following numbers within the range are specifically disclosed: R=Rl+k*(Ru−Rl), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present disclosure. Thus, the claims are a further description and are an addition to the embodiments of the present disclosure. The discussion of a reference is not an admission that it is prior art to the present disclosure, especially any reference that may have a publication date after the priority date of this application. The disclosures of all patents, patent applications, and publications cited herein are hereby incorporated by reference, to the extent that they provide exemplary, procedural, or other details supplementary to those set forth herein.

### Example 3

### Studies with Mesenchymal Stem Cells

Human bone marrow contains hematopoietic cells that differentiate to become the normal erythrocytes, leukocytes and platelets found in the blood. In addition, bone marrow contains stem-like cells that are precursors of nonhematopoietic tissues. These precursors of nonhematopoietic tissues were initially referred to as plastic-adherent cells or fibroblastic colony-forming-units because of their ability to stick to tissue culture dishes and to form colonies from single cells when grown in culture. They are currently referred to as either human mesenchymal stem cells or human multipotential stromal cells (hMSCs). These cells have attracted interest because of their potential for differentiation into a variety of tissues, such as cartilage, bone, fat and nerve, and thus, their possible use for both cell and gene therapy. There is a subpopulation of cells that have been identified in cultures of hMSCs that are small, proliferate rapidly, and undergo cyclical renewal through 3 to 4 passages when replated at low density. The small cells are precursors of more mature cells in the same cultures. These cells are referred to as rapidly self-renewing (RS) cells. RS cells retain their ability to generate single-cell derived colonies and retain their multipotentiality for differentiation.

Human mesenchymal stem cells (hMSCs; multipotent stromal cells) were obtained from the MSC distribution Center at the Texas A&M Health Science Center, Temple, Tex. The cells were grown in hMSC growing media according to the instructions from the supplier.

### Animal Studies:

hMSCs (nearly 4 million) were intravenously given to anesthetized male Sprague-Dawley rats. This was followed by the induction of hemorrhagic shock. Mesenteric post-capillary venules were observed under an intravital microscope for FITC-albumin extravasation into the extravascular space. hMSC treatment attenuated HS-induced vascular hyperpermeability significantly from 10 minutes to 60 minutes of reperfusion (p<0.05).

hMSCs Attenuate BAK-Induced Monolayer Permeability:

Rat lung microvascular endothelial cells (RLMEC) were grown as monolayers for 72 hours in fibronectin coated Transwell plates. Prior to growing RLMEC, the lower chamber of the transwell plates were seeded with hMSCs for 72 hours. After this time period, monolayers were transfected with BAK peptide (5 μg/ml) for 1 hour. Following this, FITC-albumin (5 mg/ml) was added to the luminal (upper) chamber of the Transwell and allowed to equilibrate for 30 minutes. The samples (100 μl) collected from the abluminal (lower) chambers were analyzed for FITC fluorescent intensity using a fluorometric plate reader at excitation 494 nM and 520 nM and the data were calculated as percentage of the control values. The monolayers that had hMSC grown on the lower chamber showed attenuation of BAK-induced hyperpermeability significantly (p<0.05).

hMSCs Conditioned Media Attenuates Shock Serum-Induced Monolayer Permeability:

The RLMEC monolayers were exposed to 100 μA of hMSC conditioned media for 3 hours. hMSC conditioned media was collected by layering mineral oil over confluent dishes for 18 hours. After this time period, monolayers were exposed to shock serum for 1 hour. Following this, FITC-albumin (5 mg/ml) was added to the luminal (upper) chamber of the Transwell and allowed to equilibrate for 30 minutes. Untreated and regular hMSC media treated monolayers were used as controls. The samples (100 μl) collected from the abluminal (lower) chambers were analyzed for FITC fluorescent intensity using a fluorometric plate reader at excitation 494 nM and 520 nM and the data were calculated as percentage of the control values. The monolayers that were treated with conditioned media showed attenuation of shock serum-induced hyperpermeability significantly (p<0.05).

### Stem Cell Factor on Adherens Junction Damage:

Rat lung microvascular endothelial cells were grown on fibronectin coated chamber slides in complete MCDB-3 media for 24 hours. The cells were pre-treated with SCF (100 ng/ml) for 1 hour. The cells exposed to shock serum or were transfected with caspase-3 (5 μg/ml) for 60 minutes. Caspase-3 were exposed to TransIT (10 μl/ml) for 15 minutes before exposure to the cells. The cells were washed in PBS, permeabilized with Triton X-100 and fixed with 4% paraformaldehyde. The cells were then washed in PBS, blocked with 2.5% BSA-PBS and exposed to polyclonal antibody against β-catenin overnight at 4° C. The cells were washed, mounted in an antifade-DAPI mountant and visualized utilizing a fluorescent microscope. The cells that were treated with SCF showed protection against shock serum-induced adherens junction disruption determined based on beta catenin immunofluorescnece. However, SCF did not protect adherens junctions against caspase-3 mediated disruption.

### REFERENCES

The following references, to the extent that they provide exemplary procedural or other details supplementary to those set forth herein, are specifically incorporated herein by reference.
U.S. Pat. Nos. 5,486,359; 5,827,735; 5,811,094; 5,736,396; 5,837,539; 5,837,670; 5,827,740
Case et al., Proc. Natl. Acad. Sci. USA, 96:2988-2993, 1999
Cassiede et al., J. Bone Miner. Res., 11(9): 1264-1273, 1996.
Childs et al., Shock, 29(5) 636-641, 2008.
Childs et. al. Am J. Physiol Heart Circ Physiol., 294:H2285-2295, 2008b.
Evans et al., Hum. Gene Ther., 10:1479-1489, 1999.
Fridenshtein, Arkh. Patol., 44:3-11, 1982.
Gronthos, Blood, 84(12): 4164-4173, 1994.
Jaiswal et al., J. Cell Biochem., 64(2): 295-312, 1997.
Johnstone et al., 238(1): 265-272, 1998.
Makino et al., J. Clin. Invest., 103(5): 697-705, 1999.
Miyoshi et al., Science, 283:682-686, 1999.
Sutton et al., J Virol., 72:5781-5788, 1998.
Tharakan et al., Shock, 30(5) 571-577, 2008
Tharakan et al., Shock, 66(4):1033-1039, 2009.
Uchida et al., Proc. Natl. Acad. Sci. USA, 95:11939-11944, 1998.
Yoo et al., J. Bone Joint Surg. Am., 80(12): 1745-1757, 1998.

What is claimed is:

1. A method for treating or preventing hemorrhagic shock in a subject comprising administering a composition comprising an effective amount of stem cells or a soluble factor produced by stem cells to the subject.

2. The method of claim 1, comprising administering an effective amount of stem cells to the subject.

3. The method of claim 2, wherein the stem cells are mesenchymal stem cells.

4. The method of claim 2, wherein the stem cells express elevated levels of an anti-apoptotic protein.

5. The method of claim 4, wherein the anti-apoptotic protein is a Bcl family protein.

6. The method of claim 4, wherein the anti-apoptotic protein is a recombinant protein or a protein expressed from a recombinant vector.

7. The method of claim 1, comprising administering an effective amount of a soluble factor produced by stem cells to the subject.

8. The method of claim 7, wherein the soluble factor is Stem Cell Factor (SCF).

9. The method of claim 8, wherein the SCF is recombinant.

10. The method of claim 1, where the composition further comprises a recombinant anti-apoptotic Bcl family protein.

11. The method of claim 10, wherein the recombinant anti-apoptotic Bcl family protein comprises the amino acid sequence of SEQ ID NO: 1, SEQ ID NO: 2 or a fragment thereof.

12. The method of claim 1, wherein the composition further comprises an antioxidant, a mitochondrial modulator, an endothelial growth factor, or combinations thereof.

13. The method of claim 12, wherein the endothelial growth factor elicits gene activation, cell proliferation, cell differentiation, matrix dissolution stimulation of regulatory cascades leading to angiogensis, cellular migration, degradation of matrix metalloproteinase (MMP), or combinations thereof.

14. The method of claim 12, wherein the antioxidant comprises ascorbic acid, glutathione, uric acid, carotenoids, α-tocopherol, ubiquinol, diprenyl, or combinations thereof.

15. The method of claim 12, wherein the mitochondrial modulator comprises an immunomodulatory agent, Cyclosporin A, Tacrolimus or combinations thereof.

16. The method of claim 1, wherein the composition further comprises a pharmaceutically acceptable carrier or excipient.

17. The method of claim 1, further comprising co-administering a conventional treatment for hemorrhagic shock.

18. The method of claim 17, wherein the conventional treatment for hemorrhagic shock is administration of plasma.

19. A composition comprising stem cells, which express elevated levels of an anti-apoptotic protein.

20. The composition of claim 19, wherein the anti-apoptotic protein is a Bcl family protein.

21. The composition of claim 19, wherein the anti-apoptotic protein is a recombinant protein or a protein expressed from a recombinant vector.

22. The composition of claim 19, where the anti-apoptotic protein is Bcl-xL, MCL-1, A-1 or Bcl-w.

23. The composition of claim 22, wherein the anti-apoptotic protein comprises the amino acid sequence of SEQ ID NO: 1, SEQ ID NO: 2 or a fragment thereof.

24. An article of manufacture comprising the composition of claim 19.


SEQUENCE LISTING (2 sequences)

SEQ ID NO: 1 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 2 - protein, 236 aa, Rattus norvegicus
MAQAGRTGYDNREIVMKYIHYKLSQRGYEWDTGDEDSAPLRAAPTPGIFSFQPESNRTPAVHRDTAARTSPLRPLVANAGPALSPVPPVVHLTLRRAGDDFSRRYRRDFAEMSSQLHLTPFTARGRFATVVEELFRDGVNWGRIVAFFEFGGVMCVGSVNREMSPLVDNIALWMTEYLNRHLHTWIQDNGGWDAFVELYGPSMRPLFDFSWLSLKTLLSLALVGACITLGAYLGHK